(19) United States
(12) Patent Application Publication
(10) Pub. No.: US 2010/0071094 A1
(43) Pub. Date: Mar. 18, 2010

(54) PROMOTER EXPRESSED IN THE DEHISCENCE ZONE OF DRY FRUITS

(76) Inventor: Bertrand Dubreucq, L'ECOLE (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/305,530

(22) PCT Filed: Jul. 6, 2007
(86) PCT No.: PCT/FR2007/001155

§ 371 Date: Jul. 9, 2009

(30) Foreign Application Priority Data
Jul. 6, 2006 (FR) 06/06145

(51) Int. Cl. A01H 5/00; C12N 15/82; C07H 21/00; C12N 15/63; C12N 5/04

(52) U.S. Cl. 800/290; 800/298; 800/306; 536/23.1; 536/24.1; 435/320.1; 435/419

(57) ABSTRACT

The invention relates to a promoter specific for cells of the dehiscence zone of dry fruits, and to the use of this promoter for specifically expressing a gene of interest in this dehiscence zone.

The invention relates to a promoter which allows the specific expression of a gene of interest in the cells of the dehiscence zone of dry fruit.

During evolution, plants have set up various systems for disseminating their seeds. For plants with dry dehiscent fruit, the seeds contained in the fruit are disseminated at maturity after spontaneous opening of said fruit. This opening takes place via one or more dehiscence zone(s).

A dehiscence zone consists of a zone of nonlignified cells, hereinafter referred to as rupture zone, between two zones of cells (hereinafter referred to as margins) which become lignified during maturation of the fruit; the strain induced by this lignification causes the adjacent rupture zone to tear. Hydrolytic enzymes in the cell walls could also be involved in the rupturing of the dehiscence zone.

Various types of dry dehiscent fruit exist. Mention will in particular be made of pods and siliques, which are respectively encountered in the Fabaceae and the Brassicaceae, families which contain many plants of agronomic interest.

A pod is a dry dehiscent fruit originating from a single carpel closed by means of a suture on the placental margins; it opens by means of two dehiscence zones, one located at the placental suture and the other at the median rib of the carpel.

A silique is a dry dehiscent fruit originating from two carpels, also called valves, welded together at their margins and separated by a partition, the replum. The dehiscence zones are located along the junction between the valves and replum. Each dehiscence zone comprises a rupture zone bordered on the one hand by the lignified cells of the valve margins, and on the other hand by the lignified cells of the replum. Tearing at this zone causes separation of the valves and the replum and opening of the silique.

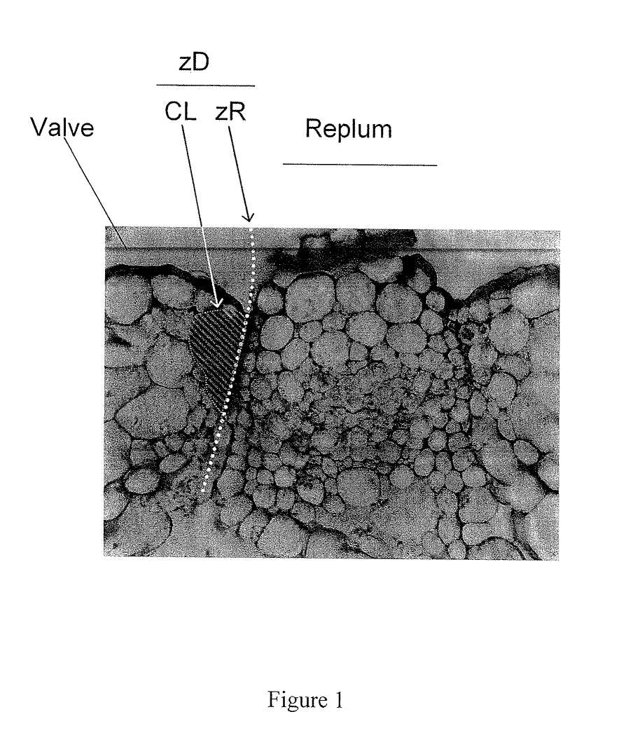

FIG. 1 represents a transverse section of a silique of Arabidopsis thaliana, indicating the positions of the valves and of the replum; the position of the rupture zone (zR), and that of the lignified cells (CL) of the valve margins has also been plotted on the left part of the figure.

Spontaneous dehiscence, which occurs when the fruit reach maturity, poses various problems in large crop plants such as, for example, rapeseed or soybean.

The main unfavourable consequence of spontaneous dehiscence is a considerable loss of yield. This is because some of the seeds fall to the ground when the siliques open and are not therefore harvested when the harvesting machine passes, causing not only a considerable loss of yield (from 10 to 25%), but also regrowth problems, which impair crop rotation. Moreover, indehiscence of pods or siliques could also make it possible to reduce infestations by certain pests or diseases in certain plants such as soybean or lentils (ROBERTS et al, Annals of Botany, 86, 223-235, 2000).

Several approaches have been proposed in order to overcome the problems posed by spontaneous dehiscence. Some concern crop practices, for example improving harvesting machines. Others have been based on the search for varieties in which there is less silique dehiscence at maturity. This approach has, for example, been proposed for rapeseed. Since few genetic variations exist with respect to this characteristic in the Brassica napus species, cultivated for the production of oil, attempts have been made to introgress one or more genes responsible for reduced silique dehiscence using Brassica rapa, in which the genetic variations for this characteristic are greater. However, it was observed that other characteristics that may be unfavourable were introgressed simultaneously.

One approach which is currently the subject of great interest consists in modifying, by genetic engineering, the expression of genes involved in the dehiscence of dry fruit, and in particular of siliques, in order to control said dehiscence.

Various genes involved in silique dehiscence have been identified (for review, cf. for example, FERRANDIZ, J of Exp. Botany 53, 377, 2031-2038, 2002).

In many cases, they are genes involved in the differentiation of the cells of the dehiscence zone. In this category, mention will be made, for example, of the SHATTERPROOF 1 (Sh1) and SHATTERPROOF 2 (Sh2) genes, encoding MADS-box transcription factors, which are involved in the lignification of the margins of the valves adjacent to the rupture zone (LILJEGREN et al Nature, 404: 766-770, 2000). When these genes are inactivated, no lignification of the valve margins or formation of the dehiscence zone are observed.

The FRUITFULL gene (Ful), which is another MADS-box transcription factor, expressed in the valves, negatively controls the expression of Sh1 and Sh2 (FERRANDIZ et al., Science, 289: 436-438, 2000). When it is overexpressed, a phenotype similar to that resulting from the inactivation of these two genes is observed. Conversely, the AGAMOUS gene (AG) positively controls the expression of Sh1 and Sh2.

The ALCATRAZ gene (Alc) is a bHLH (basic Helix-Loop-Helix) domain protein which enables the specification of the nonlignified zone at which the tearing of the dehiscence zone occurs (RAJANI and SUNDARESAN, Curr Biol 11, 1914-1922, 2001). In mutants in which this gene is inactivated, this nonlignified zone is absent, resulting in indehiscence of the fruit.

The REPLUMLESS gene (Rpl) is a homeodomain gene which is involved in the placing of the replum (ROEDER et al., Curr Biol, 13, 1630-5, 2003). Another homeodomain transcription factor, INDEHISCENT (Ind), is also known to be involved in the differentiation of the dehiscence zone (LILJEGREN et al., Cell, 116, 843-53, 2004).

Another category of genes involved in dehiscence contains genes encoding enzymes that participate in hydrolysis of the cell wall. For example, it has been observed that a polygalacturonase (PG), involved in pectin degradation, is expressed in the dehiscence zone (JENKINS et al., J. Exp. Botany 47, 1111-1115, 1996; PETERSEN et al., Plant Mol Biol, 31, 517-527, 1996).

In order to control dehiscence, it has been proposed to act on the expression of various genes involved in this dehiscence.

By way of examples: PCT Application WO 97/13865 proposes, in order to delay dehiscence, expressing, in the cells of the dehiscence zone, either an RNA which inhibits the expression of a hydrolytic enzyme of the cell wall, or a protein which interferes with the metabolism, the physiology or the development of said cells; PCT Application WO 01/79517 proposes delaying dehiscence by inhibiting the expression of the INDEHISCENT gene in the dehiscence zone.

A conventional way of acting on the expression of a gene, or the activity of the product thereof, is to express, in the same cell, an exogenous DNA sequence of which the transcription or translation product intervenes positively or negatively, depending on the application envisaged, on the expression or the activity of said protein. By way of examples, mention will be made of the overexpression of the gene in question, or of a homologue thereof, or of an activator of the expression of this gene, or, conversely, antisense suppression techniques, or RNAi techniques which are commonly used to modulate the expression of target genes.

In a large number of cases, it is preferable to target the expression of this exogenous DNA sequence to certain cells and certain tissues, in order to avoid any interference with other functions of the plant. Thus, in order to act on the dehiscence of the silique, it is desirable to have promoters specific for the various cell types making up the silique, and in particular of the dehiscence zone.

The inventors have isolated, from Arabidopsis thaliana, a promoter hereinafter referred to as pDRQ39 promoter, and have expressed the uidA (GUS) reporter gene encoding β-glucuronidase under the control of this promoter. They have thus observed that the expression of the reporter gene is restricted to the lignified cells of the silique valve margins.

This pDRQ39 promoter is located in a 361 by region upstream of the translation initiation codon of the At2g27220 gene of Arabidopsis thaliana. The sequence of this region is the following (SEQ ID NO: 1).

AGTACATATTTTTCCCATTTATGAAAAGTCCAATCACATTTTATTTATAA
AGGCGACTTATTGGACTTTCTAGTTGACAGATCAATGAAGCAGAGGATAT
TTAGATGATCATCGCAAGCAGAGAGAAAGACACGTCGAAGAAGACAATGA
GAAACTCACACCCATAATTTGCTGCTCATCATTTCTAATTATTAAATAAA
AACGGTAATTTAATTTTCTTGATTTGTCACATCTTTATTTCATCATCTTT
TGCTTACCTTTTCTTCCTAAGCTCTTGGATATTATTAATGTTTTAGATCG
ATTTATATTATAATTTGATATTTTCTTGAATTGCAAGTTTTGTTGCTTAA
TTGTCAGAATA

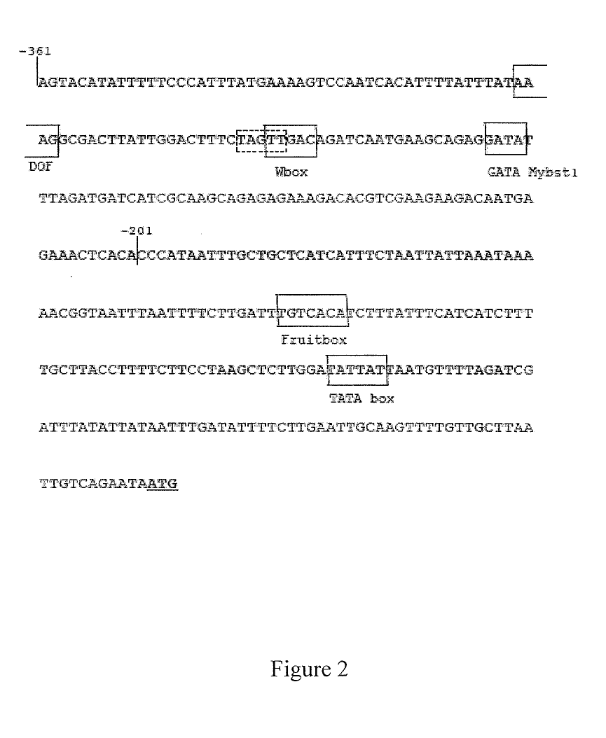

This sequence is also represented in FIG. 2, which also indicates the principal predicted boxes that may be involved in regulating the expression of this gene.

The inventors have localized the portion of this promoter essential for obtaining specific expression in the lignified cells of the silique margins in the region from nucleotide −361 to nucleotide −201 relative to the translation initiation codon of the At2g27220 gene (these positions correspond to positions 1 to 161 of the sequence SEQ ID NO:1).

In the context of the present disclosure, when a sequence is defined with reference to a range delimited by the positions of two nucleotides, it is considered that the nucleotide whose position is indicated first is included in this sequence, and the nucleotide whose position is indicated second is not included in this sequence.

Thus, the sequence ranging from position −361 to position −201 upstream of the ATG of the At2g27220 gene is the following (SEQ ID NO:2):

AGTACATATTTTTCCCATTTATGAAAAGTCCAATCACATTTTATTTATAA
AGGCGACTTATTGGACTTTCTAGTTGACAGATCAATGAAGCAGAGGATAT
TTAGATGATCATCGCAAGCAGAGAGAAAGACACGTCGAAGAAGACAATGA
GAAACTCACA

A subject of the present invention is an isolated polynucleotide that can be used for the construction of a promoter specific for the lignified cells of the margins bordering the rupture zone of a dry dehiscent fruit, which polynucleotide is characterized in that:
it has at least 80% identity, preferably at least 90% identity, and entirely preferably at least 95% identity with the polynucleotide of sequence SEQ ID NO:2;
it contains: a motif of sequence TAGTT, partially overlapping a “Wbox” of sequence TTGAC; a “DOF” box of sequence AAAG; and a “GATA Mybst1” box of sequence GATA.

The TAGTT motif, advantageously, is also found in the promoters of the SHATTERPROOF 1 and 2 and INDEHISCENT genes, which have a similar tissue expression specificity, thereby suggesting that this motif is involved in the targeting of the expression of the gene in the lignified cells of the valve margins of the silique.

A subject of the present invention is also a promoter specific for the lignified cells of the margins bordering the rupture zone of a dry dehiscent fruit, characterized in that it comprises a polynucleotide in accordance with the invention, as defined above.

The expression “promoter specific for the lignified cells of the margins bordering the rupture zone of a dry dehiscent fruit” is intended to mean a promoter capable of inducing, in these cells, a level of expression that is at least 10 times higher, preferably at least 20 times higher, than that which it induces in the cells of the other tissues of the plant.

According to one preferred embodiment of a promoter in accordance with the invention, it comprises, in addition to the boxes and motifs defined above, a “fruitbox” of sequence TGTCACA. Advantageously, a promoter in accordance with the invention comprises the polynucleotide of sequence SEQ ID NO:1.

According to another preferred embodiment of a promoter in accordance with the invention, it is a chimeric promoter, in which a polynucleotide in accordance with the invention is combined with a minimal promoter.

The construction of chimeric promoters that are functional in plants is well known in itself; a minimal promoter is combined with heterologous regulatory elements chosen according to the expression characteristics (specificity, inducibility, expression level) that it is desired to obtain. A minimal promoter comprises at least the sequences for initiation of transcription by RNA polymerase II. For example, a minimal promoter comprises, in general, in addition to a transcription initiation site, at least one TATA box located approximately 20 to 40 by upstream of the transcription initiation site, and/or at least one INR (INitiatoR) element located at the level of the transcription initiation site.

The construction of chimeric promoters by combining a minimal promoter with various regulatory elements in cis is described, for example, in U.S. Pat. No. 6,555,673, or else in the publications by RUSHTON et al. (Plant. Cell., 14, 749-762, 2002), by BHULLAR et al. (Plant. Physiology., 132, 988-998, 2003) or by DEABEAUJON et al. (Plant Cell, 15, 2514-2531, 2003).

A subject of the present invention is also the use of a promoter in accordance with the invention for specifically expressing a polynucleotide of interest in the lignified cells of the margins bordering the rupture zone of a dry dehiscent fruit.

The invention also encompasses:
the recombinant expression cassettes comprising, in addition to a promoter in accordance with the invention, a heterologous sequence of interest placed under the transcriptional control of said promoter (or one or more restriction site(s) allowing the insertion of said sequence of interest);
the recombinant vectors resulting from the insertion of a promoter or of an expression cassette in accordance with the invention into a host vector.

Said sequence of interest may in particular be a gene involved in the dehiscence of dry fruit, in particular of siliques. It may also be a polynucleotide for modulating the expression of one or more gene(s) involved in this dehiscence; this modulation may be carried out, for example, by antisense suppression or cosuppression, by the production of interfering RNA (iRNA). It is also possible to place under the control of a promoter in accordance with the invention, a polynucleotide for which it is desired to evaluate the effect on dehiscence.

The expression cassettes and recombinant vectors in accordance with the invention may, of course, also comprise other sequences, normally used in constructs of this type. The choice of these other sequences will be made, conventionally, by those skilled in the art according in particular to criteria such as the host cells chosen, the transformation protocols envisaged, etc.

By way of nonlimiting examples, mention will be made of transcription terminators, leader sequences, enhancer sequences, polyadenylation sites, etc. These sequences do not have any influence on the specificity of the promoter, but can qualitatively or quantitatively improve, overall, transcription and, where appropriate, translation.

Among the other sequences commonly used in the construction of expression cassettes and recombinant vectors, mention will also be made of sequences for following the transformation, and for identifying and/or selecting the cells or organisms transformed. They may in particular be reporter genes, which confer on these cells or organisms a readily recognizable phenotype, or else selectable marker genes.

A subject of the invention is also cells containing at least one recombinant expression cassette or one vector in accordance with the invention. They may be prokaryotic or eukaryotic cells. In the case of prokaryotic cells, they may in particular be Agrobacteria, such as Agrobacterium tumefaciens or Agrobacterium rhizogenes. In the case of eukaryotic cells, they may in particular be plant cells.

The invention also comprises transgenic plants comprising, in their genome, at least one copy of a transgene containing an expression cassette in accordance with the invention. This definition of course encompasses the progeny of the plants resulting from the initial transgenesis, provided that they conserve in their genome at least one copy of the transgene.

The plants concerned are all those which produce dry dehiscent fruit, and in particular the Brassicaceae, such as rapeseed, or the leguminous plants, such as soybean, pea, bean, etc.

The plant material (protoplasts, calluses, cuttings, seeds, etc.) obtained from these cells or plants is also part of the subject of the present invention. The invention also encompasses the products obtained from the plants in accordance with the invention, in particular the fodder, the wood, the leaves, the stems, the roots, the flowers and the fruit.

The present invention will be understood more clearly from the further description which follows, which refers to nonlimiting examples illustrating the obtaining of the pDRQ39 promoter, and the use thereof for expressing a heterologous gene.

### EXAMPLE 1

### Expression of a Reporter Gene Under the Control of Various Fragments of the Region Upstream of the At2g27220 Gene

A sequence of approximately 1500 by located 5′ of the ATG of At2g27220 was obtained from an Arabidopsis thaliana genomic DNA library.

Various fragments of this sequence were cloned, upstream of the sequence encoding the GUS reporter gene, into the pR1R2 GUS vector, by the GATEWAY™ technology using INVITROGEN kits according to the recommendations of the supplier. The recipient vector used, pR1R2 GUS (BAUDRY et al. Plant J. 39, 366-380, 2004) contains a recombination cassette LR, the coding sequence of the gene encoding β-glucuronidase from E. coli and the nos-ter terminator of the nopaline synthase gene. It also contains a kanamycin-resistance cassette.

The promoter sequences were first of all amplified by PCR using primers containing the recombination sites B1 and B2, and then introduced by BP recombination into an entry vector (pDONR 207, INVITROGEN). The recombinant vector containing the desired fragment was subsequently recombined into the pR1R2 GUS vector so as to generate the binary vectors used to transform the plants.

The constructs prepared are the following:
P1: this construct comprises the entire sequence of 1467 bp, from position −1471 to position −4 upstream of the ATG of the At2g27220 gene. The DNA was amplified using the oligonucleotides pDRQ39-1500/pDRQ39 REV.
P2: this construct comprises the sequence of 1154 bp, from position −1158 to position −4 upstream of the ATG of the At2g27220 gene. The DNA was amplified using the oligonucleotides pDRQ39-1158/pDRQ39 REV.
P3: this construct comprises the sequence of 587 bp, from position −591 to position −4 upstream of the ATG of the At2g27220 gene. The DNA was amplified using the oligonucleotides pDRQ39-590/pDRQ39 REV.
P4: this construct comprises the sequence of 356 bp, from position −360 to position −4 upstream of the ATG of the At2g27220 gene. The DNA was amplified using the oligonucleotides pDRQ39-361/pDRQ39 REV.
P5: this construct comprises the sequence of 197 bp, from position −201 to position −4 upstream of the ATG of the At2g27220 gene. The DNA was amplified using the oligonucleotides pDRQ39-201/pDRQ39 REV.

The limits of these various constructs are indicated in FIG. 2.

The oligonucleotides used are indicated below (5′-3′ orientation, the sequences in bold correspond to the Gateway recombination sequences);

pDRQ39-1500:

(SEQ ID NO: 3)

GGGACAAGTTTGTACAAAAAAGCAGGCTCGAGGAATACAGGCAGATAA
pDRQ39-1158:

(SEQ ID NO: 4)

GGGACAAGTTTGTACAAAAAAGCAGGCTGGAACCAGGCAAATAAACT
pDRQ39-590:

(SEQ ID NO: 5)

GGGACAAGTTTGTACAAAAAAGCAGGCTTCTACATTTGCGACCACATT
pDRQ39-361:

(SEQ ID NO: 6)

GGGACAAGTTTGTACAAAAAAGCAGGCTGTACATATTTTTCCCATTT
pDRQ39-201:

(SEQ ID NO: 7)

GGGACAAGTTTGTACAAAAAAGCAGGCTCCCATAATTTGCTGCTCATC
pDRQ39 REV:

(SEQ ID NO: 8)

GGGGACCACTTTGTACAAGAAAGCTGGGTCTGACAATTAAGCAACAAAAC

These various constructs were transferred into Arabidopsis thaliana plants, ecotype Wassilewskija, by the floral infiltration method (BECHTOLD et al., C.R. Acad. Sci., 316, 1194-1199, 1993). The seeds were sterilized and the primary transformants were selected on an MS medium (Duchefa, MO2 555, pH 5.6) containing kanamycin (100 mg/l), the selection agent. The plants selected were subsequently transferred into pots and grown under glass. Only the fertile plants exhibiting a normal phenotype were retained for the analysis.

gus gene expression was sought in the vegetative organs and in the siliques and seeds of the T1 and T2 generations.

The β-glucuronidase activity was detected by means of a histochemical test, by detecting the blue coloration resulting from the hydrolysis of 5-bromo-4-chloro-3-indolyl-β-D-glucuronic acid (X-gluc according to the protocol described by JEFFERSON et al (Plant Mol. Biol. Report 5, 387-405, 1987)).

### gus Gene Expression in the Vegetative Organs:

The analysis was carried out on leaves, and stems under glass, and on roots in vitro. No gus activity is detected in the nontransformed plants. In the case of the plants transformed with the P1, P2, P3, P4 and P5 constructs, no activity is detected after incubation in the presence of X-Gluc. These results show that the At2g27220 promoter does not allow expression of the gus gene in the organs tested.

### GUS Gene Expression in the Silique and the Seed

In order to determine the localization of the expression of the GUS gene under the control of the At2g27220 promoter, histological sections were cut and observed by photon microscopy.

With the P1, P2, P3 or P4 constructs, considerable labelling is observed in the silique valve margins, whereas the cells of the embryo, of the replum and of the mesocarp show no labelling. For the P5 construct, no labelling is detected, suggesting that said construct no longer contains the necessary regulatory elements.

### EXAMPLE 2

### Structural Analysis of the Promoter of the At2g27220 Gene

The promoter of the At2g27220 gene was analysed in order to search for putative regulatory motifs that may be involved in controlling the expression.

The promoter was compared with the promoter sequences of the SHATTERPROOF 1 and SHATTERPROOF 2 genes (At3g58780, At2g42830, respectively) and the INDEHISCENTI gene (At1g00120) of Arabidopsis thaliana.

This analysis shows the existence of a 5 by motif (TAGTT) common to these promoters, and absent from the promoters of other transcription factors expressed in cells adjacent to the dehiscence zones.

Among the other putative transcription regulating sequences identified in the pDRQ39 promoter, the following will in particular be pointed out: a “DOF” box (YANAGISAWA and SCHMIDT, Plant J 17, 209-214 1999); a “DATA Mybst 1” box (TEAKLE et al., Plant Mol Biol. 50, 43-57 (2002); a “W” box (CHEN et al., Plant Cell 14, 559-574, 2002), located between −361 and −201. A “Fruit box” (TGTCACA motif; YAMAGATA et al., J Biol Chem. 277, 11582-11590, 2002) was also identified in the region located between −201 and the translation initiation site. This box could play a role in modulating the boxes located between −361 and −201.

The 1467 by sequence upstream of the translation initiation codon of the At2g27220 gene is represented in FIG. 2. The translation initiation site is represented in bold and underlined. The “DOF”, “GATA”, “W” boxes and “Fruit box” are boxed-in in solid lines. The TAGTT motif is boxed-in in dashed lines.

1. Isolated polynucleotide that can be used for the construction of a promoter specific for the lignified cells of the margins bordering the rupture zone of a dry dehiscent fruit, which polynucleotide is characterized in that:
it has at least 80% identity with the polynucleotide of sequence SEQ ID NO:2;
it contains: a motif of sequence TAGTT, partially overlapping a “Wbox” of sequence TTGAC; a “DOF” box of sequence AAAG; and a “GATA” box of sequence GATA.

2. Isolated promoter which allows the specific expression of a polynucleotide of interest in the lignified cells of the margins bordering the rupture zone of a dry dehiscent fruit, characterized in that it comprises a polynucleotide according to claim 1.

3. Promoter according to claim 2, characterized in that it comprises the polynucleotide sequence SEQ ID NO:1.

4. Chimeric promoter containing a polynucleotide according to claim 1, combined with a minimal promoter.

5. A method for specifically expressing a polynucleotide of interest in the lignified cells of the margins bordering the rupture zone of a dry dehiscent fruit, which comprises utilizing the isolated polynucleotide according to claim 1.

6. Recombinant expression cassette, characterized in that it comprises a promoter according to claim 1, and a gene of interest placed under the transcriptional control of said promoter.

7. Recombinant vector comprising an expression cassette according to claim 6.

8. Host cell transformed with an expression cassette according to claim 6.

9. Host cell according to claim 8, characterized in that it is a plant cell.

10. Transgenic plant comprising at least one transgene containing an expression cassette according to claim 6.

11. Transgenic plant according to claim 10, characterized in that it is a plant of the family Brassicaceae or a cruciferous plant.

12. The isolated polynucleotide according to claim 1, wherein it has at least 90% identity with the polynucleotide of sequence SEQ ID NO:2.

13. The isolated polynucleotide according to claim 1, wherein it has at least 95% identity with the polynucleotide of sequence SEQ ID NO:2.


SEQUENCE LISTING (8 sequences)

SEQ ID NO: 1 - DNA, 361 bp, Arabidopsis thaliana
agtacatatttttcccatttatgaaaagtccaatcacattttatttataaaggcgacttattggactttctagttgacagatcaatgaagcagaggatatttagatgatcatcgcaagcagagagaaagacacgtcgaagaagacaatgagaaactcacacccataatttgctgctcatcatttctaattattaaataaaaacggtaatttaattttcttgatttgtcacatctttatttcatcatcttttgcttaccttttcttcctaagctcttggatattattaatgttttagatcgatttatattataatttgatattttcttgaattgcaagttttgttgcttaattgtcagaata

SEQ ID NO: 2 - DNA, 160 bp, Arabidopsis thaliana
agtacatatttttcccatttatgaaaagtccaatcacattttatttataaaggcgacttattggactttctagttgacagatcaatgaagcagaggatatttagatgatcatcgcaagcagagagaaagacacgtcgaagaagacaatgagaaactcaca

SEQ ID NO: 3 - DNA, 48 bp, Artificial
  PCR primer
gggacaagtttgtacaaaaaagcaggctcgaggaatacaggcagataa

SEQ ID NO: 4 - DNA, 47 bp, Artificial
  PCR primer
gggacaagtttgtacaaaaaagcaggctggaaccaggcaaataaact

SEQ ID NO: 5 - DNA, 48 bp, Artificial
  PCR primer
gggacaagtttgtacaaaaaagcaggcttctacatttgcgaccacatt

SEQ ID NO: 6 - DNA, 47 bp, Artificial
  PCR primer
gggacaagtttgtacaaaaaagcaggctgtacatatttttcccattt

SEQ ID NO: 7 - DNA, 48 bp, Artificial
  PCR primer
gggacaagtttgtacaaaaaagcaggctcccataatttgctgctcatc

SEQ ID NO: 8 - DNA, 50 bp, Artificial
  PCR primer
ggggaccactttgtacaagaaagctgggtctgacaattaagcaacaaaac